(19) United States
(12) Patent Application Publication
(10) Pub. No.: US 2011/0073672 A1
(43) Pub. Date: Mar. 31, 2011

(54) Fragrant apparatus

(76) Inventor: KEN CHEN, Rangeville (AU)

(21) Appl. No.: 12/567,793

(22) Filed: Sep. 27, 2009

(51) Int. Cl. A61L 9/01; B05B 15/00

(52) U.S. Cl. 239/55; 239/289

(57) ABSTRACT

A fragrant apparatus includes a container combined with the top of a retaining base, a fragrant solution is filled into the container, a volatile medium contained in the container, and a light emitting device installed at the bottom of the retaining base for providing a lighting decorative effect.

### BACKGROUND OF THE INVENTION

1. Field of the Invention

The present invention relates to a fragrant apparatus integrated with modern electronic and fragrant functions.

2. Description of the Related Art

Fragrance generation products available in the market can be roughly divided into the following three types:

Fragrant products of the first type including scented candles and scent pots, etc require a fire source for burning the fragrant products to disperse fragrance, and producing a light the decorative effect from the burning fire source.

Fragrant products of the second type are connected electrically for converting electric energy into heat energy to heat up a fragrant matter and disperse fragrance, and its effect of dispersing fragrance is more significant than that of the first type of fragrant products.

Fragrant products of the third type disperse fragrance from a fragrant matter by a capillary action, and fragrant products of this type are generally designed with a decorative effect, and a dispersion of a mild fragrant odor.

However, the fragrant products of the aforementioned first and second types produce fragrance through heat energy. Regardless of burning the fragrant products directly by a light source or converting electric energy into heat energy, the fragrant products of these type may cause a fire accident during their practical application. For example, the fragrant products of the first type may cause a fire accident easily when any flammable material is near the fire source, and the fragrant products of the second type may cause a fire accident due to electric fire or improper use. Although the fragrant products of the third type can disperse fragrance, yet the decorative effect is incomparable with the lighting decorative effect of the fragrant products of the first and second types.

It is a key point of the present invention to overcome the aforementioned problems of the conventional fragrant products.

### SUMMARY OF THE INVENTION

Therefore, it is a primary objective of the present invention to overcome the problems of the prior art by providing a fragrant apparatus for achieving a fragrant effect, while connecting to an LED control device to achieve a lighting decorative effect.

To achieve the foregoing effect, the present invention provides a fragrant apparatus comprising:

a hollow retaining base, including a transverse partition plate installed in the retaining base, an upper chamber disposed at the top of the partition plate, a lower chamber disposed at the bottom of the partition plate, and a through hole disposed at the center of the partition plate and interconnected with the upper chamber and the lower chamber;

a container, with the bottom coupled with the top of the retaining base, and including a containing space for containing a volatile fragrant solution, an opening formed on the container, at least one volatile medium inserted into the containing space from the opening for absorbing the fragrant solution, and a portion of the volatile medium being exposed from the opening; and

a light emitting device, contained into the lower chamber of the retaining base, and having a light emitting element disposed inside a casing of the light emitting device, installed at the bottom of the container, and situated at the through hole opposite to the partition plate, and electrically coupled to a control unit for controlling a light emitting status of the light emitting device.

The foregoing and other objectives and advantages of the present invention will become apparent with the detailed description of the following preferred embodiment together with the accompanying drawings as follows.

Of course, the present invention may have modifications and variations on some of the elements or the arrangement of the elements, but the selected preferred embodiments in this specification will be described in details, and their structures will be illustrated in the related drawings.

### BRIEF DESCRIPTION OF THE DRAWINGS

FIG. 1 is a perspective view of the present invention;

FIG. 2 is an exploded view of the present invention;

FIG. 3 is a cross-sectional view of the present invention;

FIG. 4 is a schematic view of an application in accordance with a first preferred embodiment of the present invention; and

FIG. 5 is a schematic view of an application in accordance with a second preferred embodiment of the present invention.

### DETAILED DESCRIPTION OF THE PREFERRED EMBODIMENTS

The technical characteristics of the present invention will become apparent with the detailed description of the following preferred embodiments and related drawings.

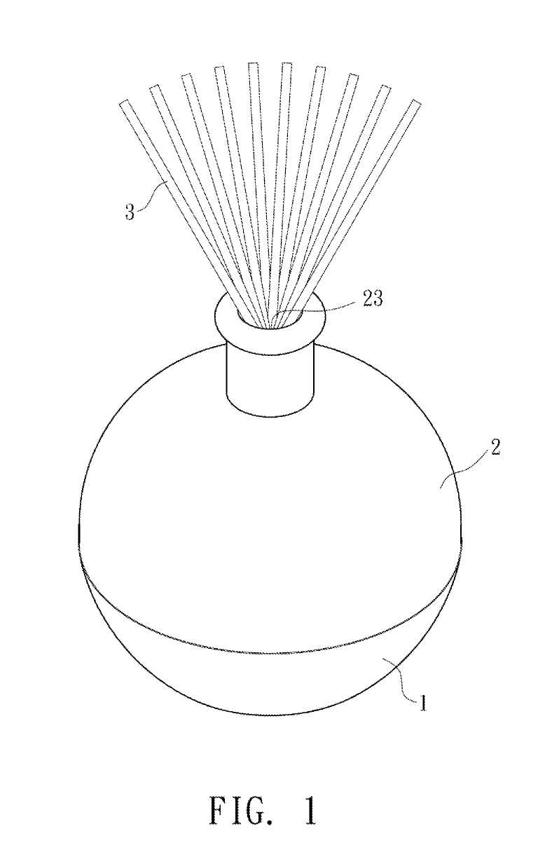

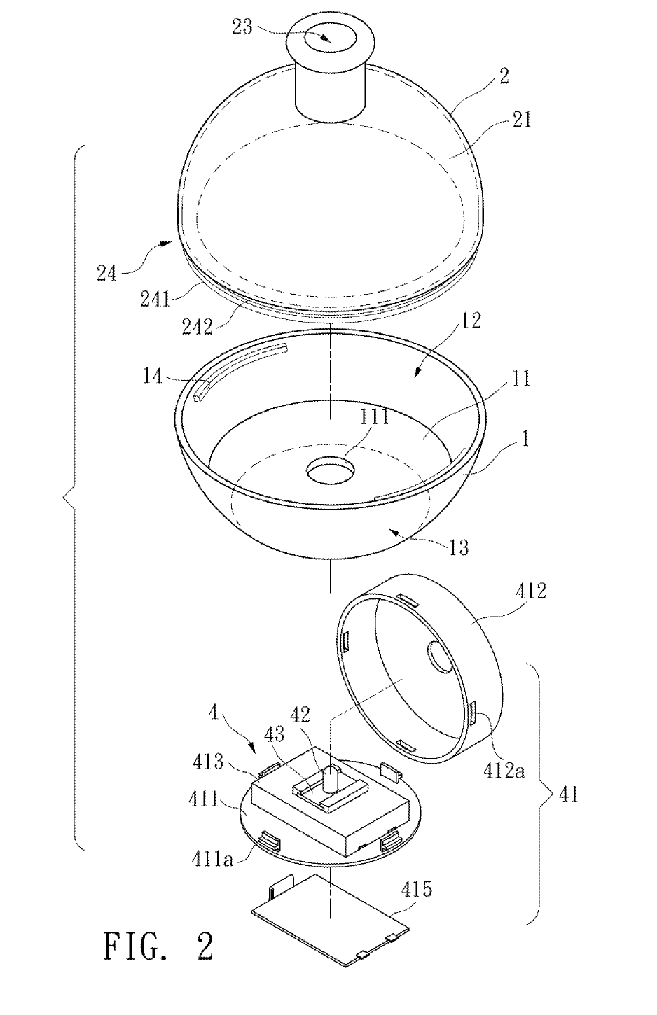

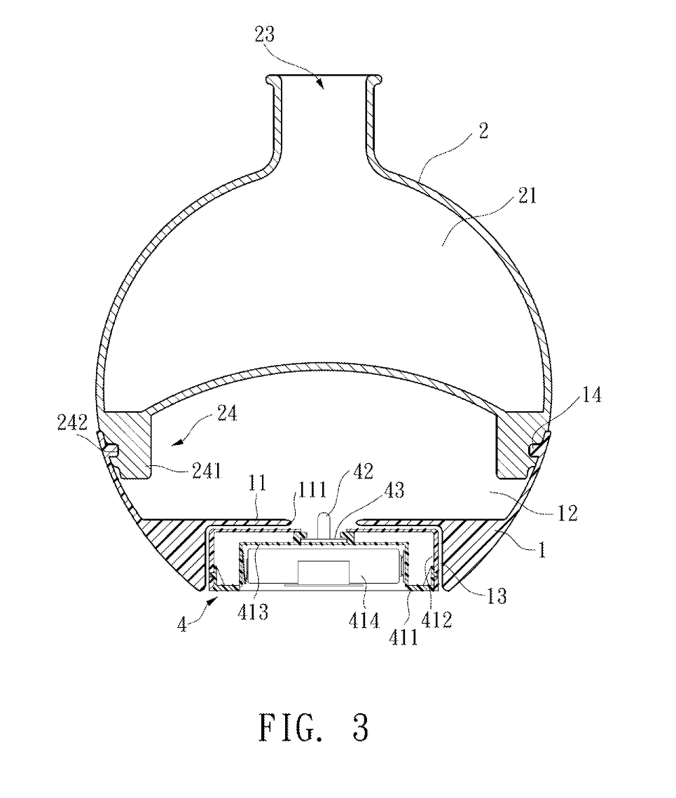

With reference to FIGS. 1 to 4 for structures of the selected preferred embodiments of the present invention, the embodiments are provided for illustration purpose only, but the invention is not limited to such structures only.

This preferred embodiment provides a fragrant apparatus, comprises the following elements:

A hollow retaining base 1 includes a transverse partition plate 11 installed in the retaining base 1, an upper chamber 12 disposed at the top of the partition plate 11 of the retaining base 1, a lower chamber 13 disposed at the bottom of the partition plate 11, and a through hole 111 disposed at the center of the partition plate 11 and interconnected with the upper chamber 12 and the lower chamber 13.

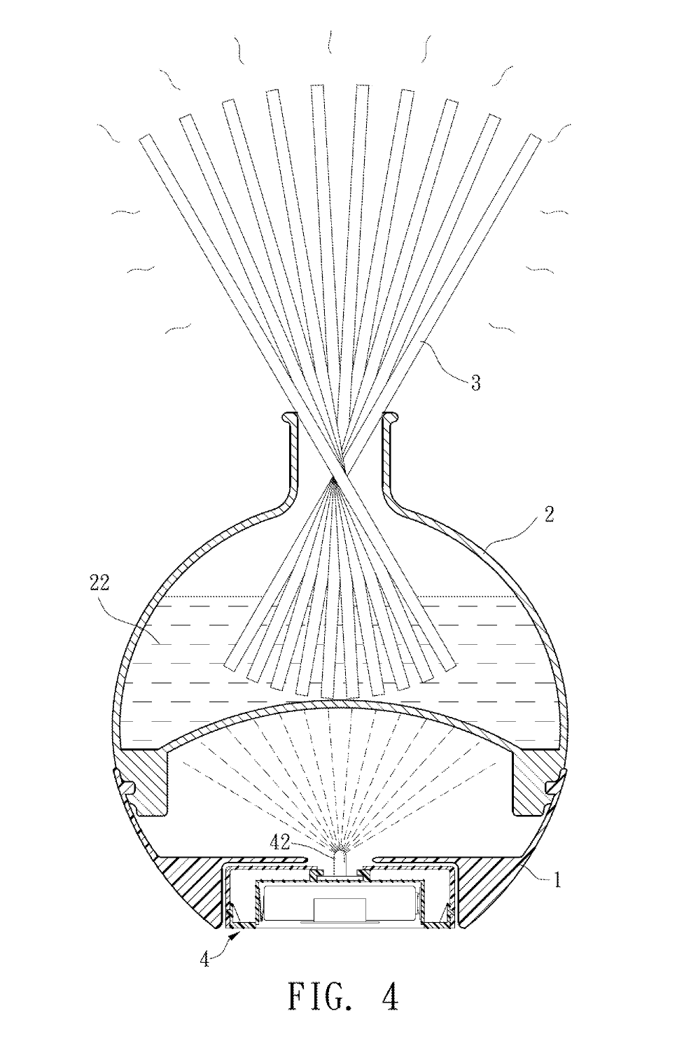

A container 2 has its bottom coupled to the top of the retaining base 1, and includes a containing space 21 for containing a volatile fragrant solution 22, and the container 2 further includes an opening 23, at least one volatile medium 3 inserted from the opening 23 into the containing space 21 for absorbing the fragrant solution, and a portion of the volatile medium 3 exposed from the opening 23, wherein the volatile medium 3 of this preferred embodiment is in the shape of a rod as shown in FIGS. 1 and 4, and a plurality of volatile media 3 are inserted into the container 2.

In this preferred embodiment, the retaining base 1 includes at least one first fixing portion 14 disposed at the top of retaining base 1, and at least one second fixing portion 24 disposed at the bottom of the container 2, and the container 2 is fixed and coupled with the first fixing portion 14 of the retaining base 1 by the second fixing portion 24. The retaining base 1 further includes a first fixing portion 14 protruded separately from two opposite internal sides towards the interior of the upper chamber 12, wherein each first fixing portion 14 is in the shape of a long bar and extended transversely, and the second fixing portion 24 has an extension portion 241 disposed downwardly from the periphery of the second fixing portion 24, and an inwardly concave portion 242 protruded from the second fixing portion 24 and corresponding to each protruded first fixing portion 14, and the first fixing portion 14 protruded from the interior of the retaining base 1 and the inwardly concave portion 242 in the extension portion 241 of the second fixing portion 24 are coupled, embedded and fixed with each other.

A light emitting device 4 is contained in the lower chamber 13 of the retaining base 1, and the light emitting device 4 includes a light emitting element 42 installed in a casing 41. The light emitting element 42 of this preferred embodiment is an LED lamp, for emitting light at the bottom of the container 2, and the light emitting element 42 is disposed in the through hole 111 opposite to the partition plate 11, passed through the through hole 111, extended slightly into the lower chamber 13, and electrically coupled to a control unit 43, and this control unit 43 is provided for controlling a light emitting status of the light emitting element 42. For example, the fragrant apparatus can be turned on by an optical sensing control method, a touch control method, a remote control method, a sound control method, or a control unit method, and the fragrant apparatus also controls a light color according to the temperature of the fragrant apparatus and a change of light color.

The casing 41 of the light emitting device 4 of this preferred embodiment includes a chassis 411 and a cover 412 covered onto the chassis 411, and the light emitting element 42 is protruded outwardly from the cover 412, and the control unit 43 is installed on the chassis 411 and electrically coupled to the light emitting element 42. The chassis 411 includes a battery compartment 413 for installing a battery 414, and the battery compartment 413 includes a battery lid 415. The cover 412 of this preferred embodiment includes a latch slot 412a and a latch 411a corresponding to each latch slot 412a in the chassis 411, and the chassis 411 and the cover 412 are coupled and fixed with each other by the latch 411a and the latch slot 412a.

From the aforementioned structure, the light emitting device 4 is installed in the lower chamber 13 of the retaining base 1, and the battery 412 is used as a power source, and the control unit 43 is provided for controlling a light emitting status of the light emitting element 42, and the light emitting element 42 is provided for emitting light such that the fragrant apparatus has a bright light, and thus provides a light decorative effect.

In summation, the present invention has the following advantages. The fragrant apparatus uses the light emitting element 42 as a light source to produce the lighting decorative effect, and thus giving better safety and decorative effects than the conventional fragrant apparatus.

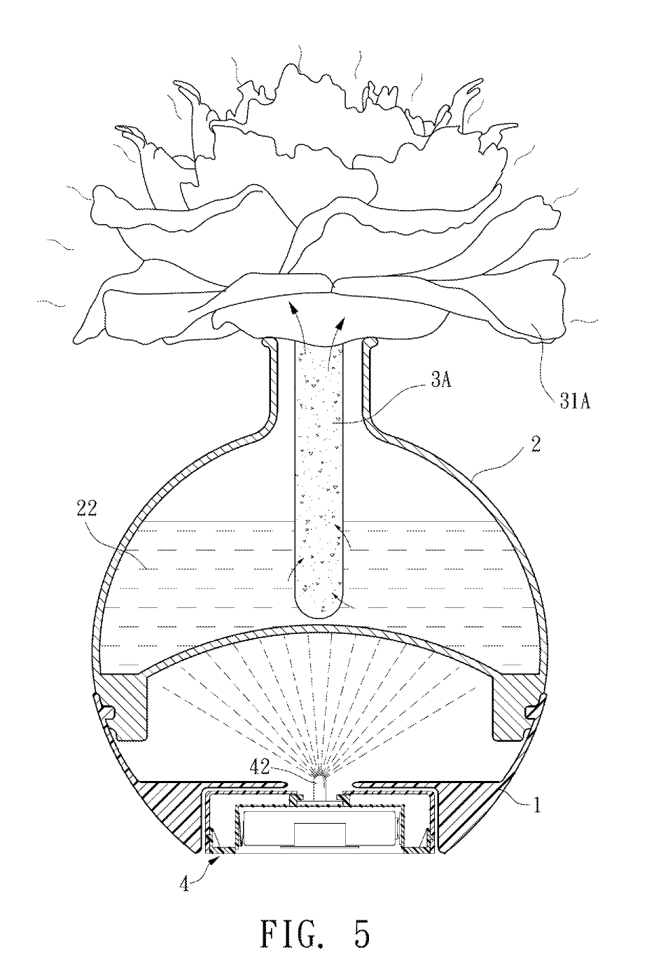

Of course, there are many other embodiments of the present invention with minor alterations and modifications. With reference FIG. 5 for a second preferred embodiment of the present invention, the volatile medium 3A is a rod, and a flower 31A made of a special material having a capillary action and installed outside the container 2 for showing different decorative modes of the fragrant apparatus of the present invention.

While the invention has been described by means of specific embodiments, numerous modifications and variations could be made thereto by those skilled in the art without departing from the scope and spirit of the invention set forth in the claims.

Persons ordinarily skilled in the art should understand that the present invention definitely can achieve the foregoing objectives. The present invention complies with the patent application requirements, and thus is duly filed for patent application.

What is claimed is:

1. A fragrant apparatus, comprising:
a hollow retaining base, having a transverse partition plate installed in the retaining base, an upper chamber disposed at the top of the partition plate, a lower chamber disposed at the bottom of the partition plate, and a through hole formed at the center of the partition plate and interconnected with the upper chamber and the lower chamber;
a container, with the bottom coupled with the top of the retaining base, and including a containing space for containing a volatile fragrant solution, an opening formed on the container, at least one volatile medium inserted into the containing space from the opening for absorbing the fragrant solution, and a portion of the volatile medium being exposed from the opening; and
a light emitting device, contained into the lower chamber of the retaining base, and having a light emitting element disposed inside a casing of the light emitting device, installed at the bottom of the container, and situated at the through hole opposite to the partition plate, and electrically coupled to a control unit for controlling a light emitting status of the light emitting device.

2. The fragrant apparatus of claim 1, wherein the retaining base includes at least one first fixing portion disposed at the top of the retaining base, at least one second fixing portion disposed at the bottom of the container, and the container is fixed with a first fixing portion of the retaining base by the second fixing portion.

3. The fragrant apparatus of claim 2, wherein the retaining base includes a first fixing portion protruded separately from two opposite internal sides of the retaining base towards the upper chamber, and each first fixing portion is in a long-bar shape and extended transversely, and the second fixing portion includes an extension portion disposed downwardly on the second fixing portion, an inwardly concave portion disposed at a position corresponding to each protruded first fixing portion, and the first fixing portion protruded from the interior of the retaining base is coupled, embedded and fixed with the inwardly concave portion in the extension portion of the second fixing portion.

4. The fragrant apparatus of claim 1, wherein the casing of the light emitting device includes a chassis and a cover covered onto the chassis, and the light emitting element and the control unit are disposed on and electrically coupled to the chassis.

5. The fragrant apparatus of claim 4, wherein the chassis includes a battery compartment for installing a battery, and the battery compartment includes a battery lid, and either one of the chassis and cover includes a latch slot disposed at the periphery, and the other one of the chassis and cover includes a latch, such that the chassis and the cover can be coupled and fixed with each other by engaging the latch and the latch slot with each other.

6. The fragrant apparatus of claim 1, wherein the control unit is turned on by an optical sensing control method, a touch control method, a remote control method, or a sound control method, and the control unit controls a light color according to the temperature of the fragrant apparatus, and controls a change of the light color.
